(19) United States
(12) Patent Application Publication
(10) Pub. No.: US 2008/0288003 A1
(43) Pub. Date: Nov. 20, 2008

(54) REVERSIBLY EXPANDABLE FIXATION DEVICE

(76) Inventor: Laurence M. McKinley, Escondido, CA (US)

Correspondence Address: KAUTH , POMEROY , PECK & BAILEY ,LLP, P.O. BOX 19152, IRVINE, CA, 92623, US

(21) Appl. No.: 11/935,193

(22) Filed: Nov. 5, 2007

Related U.S. Application Data
(60) Provisional application No. 60/864,478, filed on Nov. 6, 2006.

(51) Int. Cl. A61B 17/04

(52) U.S. Cl. 606/313; 606/301; 606/305

(57) ABSTRACT

A reversibly expandable vertebral fixation screw and minimally invasive techniques for inserting the screw are provided. The screw incorporates a divided tip and an expansion device that allows for the divided components of the tip of the screw to expanded outward to engage the internal surfaces of the vertebral body.

### CROSS-REFERENCE TO RELATED APPLICATIONS

The current invention claims priority to U.S. Provisional Patent Application No. 60/864,478, filed Nov. 6, 2006, the disclosure of which is incorporated herein by reference.

### FIELD OF THE INVENTION

The current invention is directed to a reversibly expandable fixation device for application in osteoporotic vertebral bodies.

### BACKGROUND OF THE INVENTION

Internal fixation systems have been a major step forward for patients with healthy underlying bone structure; however, such systems have proven generally unreliable for patients with osteoporosis. Specifically, the poor quality of the bone into which the anchor screws and plates are driven in these osteoporotic patients often leads to screw loosening and mechanical failure of the implant. (See, e.g., Goldhahn, et al., J. Ortho. Res. 917-925, (2006), the disclosure of which is incorporated herein by reference.) This is particularly tragic as such patients are most often in need of the type of spinal fusion such systems are meant to address.

There have been a few attempts to address the need for improved fixation systems that can compensate for the inferior holding strength in osteoporotic bone and vertebrae. One solution has been to enlarge the implant/bone interface by increasing the surface area of the fixation screw. For example, Schroeder and colleagues attempted to provide a fixation screw with improved surface area by using a hollow perforated cylinder. (See, e.g., Schroeder, et al., SSO Schweiz Monatsschr Zahnheilkd 86:713-727, (1976), the disclosure of which is incorporated by reference.) Goldhahn addressed the question of improved fixation stability by creating a three-lobed fixation screw with an elliptical cross-section to increase the periphery volume of the implant. (See, e.g., Goldhahn, et al., WIPO Pub. WO 01/80754 A1, the disclosure of which is incorporated herein by reference.) Another proposed solution has been to insert a porous cannulated screw that would allow the injection of cement around the screw thereby enhancing the stability of the bone/implant interface. (See, e.g., Fransen, J. Neurosurg. Spine 7:366-369, (2007), the disclosure of which is incorporated herein by reference.)

However, all of these methods continue to rely on the structural stability of a threaded body. Inherently such screws have a large polar moment of inertia and are therefore prone to cutting through the cancellous bone under the types of repetitive loads typically experienced in spinal fusions. Accordingly, despite the longstanding interest in finding appropriate biomechanical solutions to the problem of cervical stability in osteoporotic patients, and the extensive research conducted in the area, researchers and practioners continue to search for an internal fixation system that provides maximal interface stability between the bone and implant.

### SUMMARY OF THE INVENTION

The current invention is directed to a vertebral fixation screw for use in bone requiring additional stabilization, and more particularly to an expandable vertebral fixation screw.

In one embodiment, the expandable vertebral fixation screw comprises an expandable body capable of locking against anatomical structures within the pedicle of the vertebrae. In such an embodiment, the tip of the screw may be expandable.

In another embodiment, the screw is expanded using a central expansion rod.

In still another embodiment, the screw is expanded using a counter-rotation mechanism.

In yet another embodiment, the screw is cannulated to allow minimally invasive surgical techniques.

In still yet another embodiment, the screw is cannulated and/or porous to allow for the injection of cement or osseous integration materials into the pedicle.

The invention is also directed to a method of stabilizing a patient's spine using the expandable screws of the current invention.

### BRIEF DESCRIPTION OF THE FIGURES

These and other features and advantages of the present invention will be better understood by reference to the following detailed description when considered in conjunction with the accompanying drawings wherein:

FIG. 1a shows a schematic diagram of an exemplary expandable screw inserted into the vertebral body of a patient in an unexpanded state;

FIG. 1b shows a schematic diagram of an exemplary expandable screw inserted into the vertebral body of a patient in an expanded state;

FIG. 2a shows a schematic diagram of an exemplary expandable screw in an unexpanded state;

FIG. 2b shows a schematic diagram of an exemplary expandable screw in an expanded state;

FIG. 3a shows a schematic diagram of another exemplary expandable screw in an unexpanded state; and

FIG. 3b shows a schematic diagram of another exemplary expandable screw in an expanded state; and

FIG. 4 shows a schematic diagram of an exemplary cannulated expandable screw.

### DETAILED DESCRIPTION OF THE INVENTION

The current invention is directed to a vertebral fixation screw for use in bone requiring additional stabilization, and more particularly to an expandable vertebral fixation screw.

Prior to describing the expandable screw of the current invention it is important to understand the environment the screw is to be engaged in. Vertebral fixation screws are generally driven into the cancellous bone found in the center of the vertebral body. For the purposes of this application this cancellous bone can be imagined as a scaffolding formed of horizontal, vertical and cross support members. Between these supports are large linking molecules, which are typically calcium salts. In healthy bone there is a large concentration of these molecules and supports such that a dense network is formed that can provide the support necessary for locking a threaded screw into place once inserted into the bone. The screw is locked into place because the interlocking molecules and scaffolding prevents the network of material from racking and twisting when placed under a load.

However, in unhealthy bone, such as that encountered with osteoporotic patients, there is a 50 to 60% reduction in the concentration of both the scaffold and linking molecules in this cancellous bone. Unless some alternative support can be provided there is simply not enough density in the bone to ensure that the screw doesn't twist and cut through the weak bone once place under load.

In situations where poor bone quality is encountered a rescue screw would typically be used. For example, rescue screws have been used in situations where a screw has been inserted into the cortical tables of the skull, but does not have sufficient purchase and is therefore loose, or in the vertebrae if poor fixation has been encountered and a larger screw is not available. Currently, there are only two choices; one is to put in some bone cement, and the other would is to take out the poorly seated screw and put in a screw having a bigger diameter. However, both of these solutions still depend on anchoring the screw within the weak cancellous bone. Moreover, there is an limit to the size of screw that can be inserted into a vertebral body limiting the efficacy of such solutions.

The screw of the current invention allows for the expansion of the tip of the screw after insertion in those cases where it is not possible to anchor the screw into the cancellous bone of a vertebral body. By expanding the tip of the screw you allow for the anchorage of the screw against the far end of the pedicle shaft in the cortical bone, thus avoiding the inherent problems in anchoring into the cancellous bone of such patients. Moreover, by expanding the screw and catching the anterior portion of the pedicle, it is possible to obtain a seating strength that is greater than 50% of the maximum theoretical pullout strength. As a comparison, studies from the 1980s show that one could only obtain a 100% pullout strength if the screw were driven all the way though the front of the vertebrae. However, such a technique is never recommended as it raises the possibility that the screw could be driven into the back of the aorta, a possibly fatal complication.

In addition, by actively and mechanically anchoring the screw against the wall of the bone the current invention creates immediate stability, which allows quicker recovery for the patient and also allows the screw to become osseously integrated with the bone. The immediate stability of the screw is important because if the bone is allowed to shift within the pedicle, cleavage lines will form around the screw and its thread pattern preventing successful and complete integration with the surrounding bone.

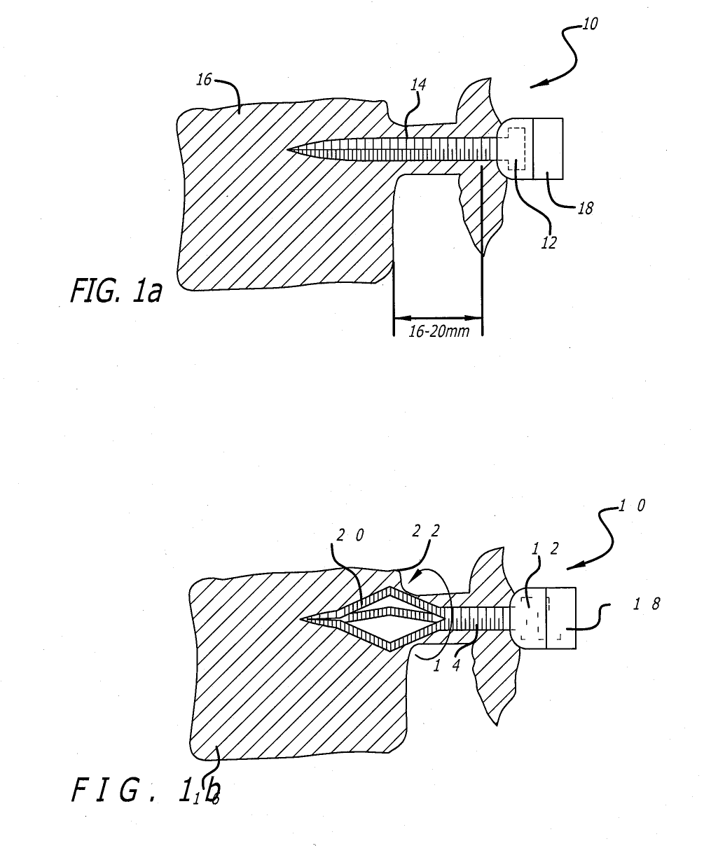

Turning to the structure of the expandable screw of the current invention, as shown in FIGS. 1a and 1b, the screw (10) generally comprises a head portion (12) for driving the screw, and an elongated threaded portion (14) for securing the screw within the vertebral body (16). The head portion (12) is itself positioned within a rod seating portion (18) that is designed for interconnecting the screw to rods, plates or other spinal fusion attachments. Although an exemplary embodiment of the invention having a “T” shaped head (12), a cannulated threaded portion (14) and a cup shaped rod seating portion (18) is shown in FIG. 1, it should be understood that none of these specific structures are critical to the operation of the current expandable screw. Specifically, any screw head design that allows for the screw to be driven into the bone and mated with a rod seating device may be used with the current invention, including a ball arrangement or even conventional tapered, mechanical, wood or metal type screw heads. Likewise, any suitable rod seating structure (18) may be used such that the head of the screw may be retained within the rod seating portion (18), and such that spinal fusion attachments may be attached to the screw through the rod seating portion.

The one feature of the screw that is critical to the operation of the current invention, as shown in FIG. 1b, is the ability to reversibly expand at least the tip (20) of the threaded portion (14) of the rescue screw. As shown in FIG. 1b, the threaded portion (14) of the screw is expanded outward to lock the outer threads of the screw against the anatomical structures of the vertebral body to ensure the greatest possible mechanical stability of the seat between the screw and the bone. For example, in the embodiment shown in FIG. 1, only the tip (20) of the threaded portion (14) is expanded such that the expanded portion of the screw seats against the distal edge of the pedicle (22). During operation, the screw is first driven into the vertebral body, as shown in FIG. 1a, and then the threaded portion is expanded outward to mechanically seat the tip of the screw against the distal pedicle of the vertebral body (22), as shown in FIG. 1b.

Turning now to the operation of the expandable screw of the current invention. It should first be understood that although two possible expansion mechanisms are discussed below, any expansion mechanism, such as a molly bolt or expansion bolt, that allows for the reversible expansion of at least the tip of the threaded portion of the screw may be used with the expandable screw of the current invention.

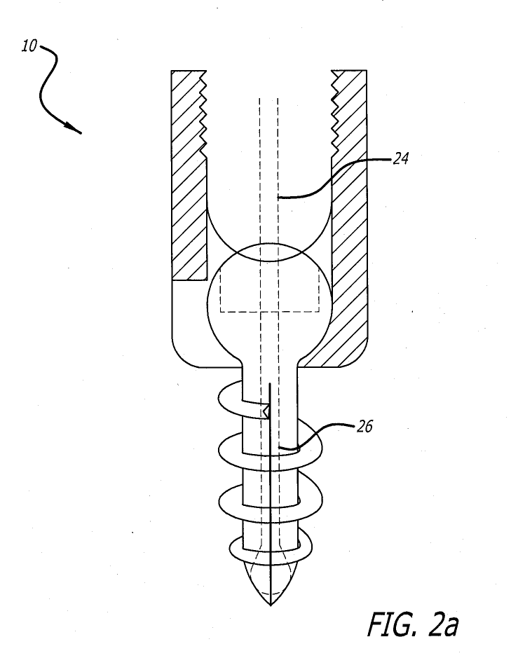

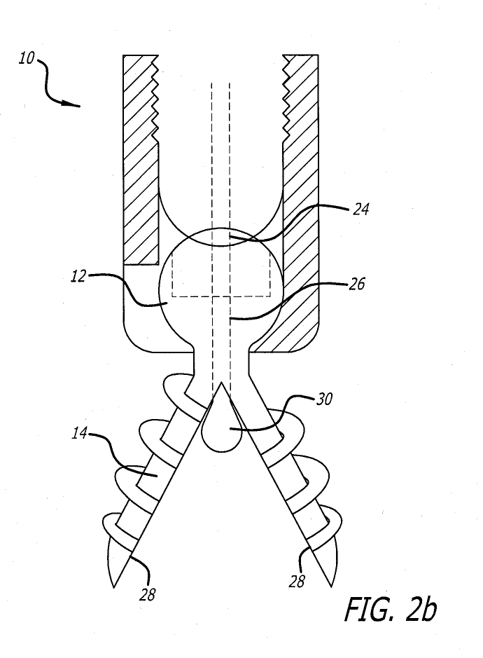

For example, in a first exemplary embodiment as shown in FIGS. 2a and 2b, the expandable screw (10) of the current invention incorporates an expansive device that comprises a mandrill (24) positioned within a central hollow portion (26) that extends the length of the threaded portion (14) of the screw. During operation, once the screw is in place the mandrill (24) can be withdrawn through the central hollow portion (26) of the cannulated screw. As shown in FIG. 2b, in such an embodiment the threaded shaft is divided into two or more components (28), and as the mandrel is pulled upward the expanded tip portion of the mandrel (30) pushes the divided components of the thread (28) outward against the surrounding walls of the vertebral body (not shown). Although only the tip of the mandrill is shown it should be understood that any portion of the mandrill may be expanded such that the radius of the expanded portion is larger than the hollow portion (26) of the screw. In such an embodiment, any mechanism may be used to force the mandrill up through the center of the screw. For example, the mandrill could be formed as shown in FIG. 2 as a simple smooth body that is pulled upward by a surgeon. Alternatively, the inside wall of the central hollow portion (26) and the outer surface of the mandrill (24) could be threaded such that the mandrill is rotated up through the central hollow portion. In such an embodiment, any suitable driver might be used to torque the mandrill, including a screwdriver or torque wrench. Once the mandrill is raised to the desired level the shaft of the mandrill (24) can be bent down or broken off at the level of the head (12) of the screw providing a flush surface. Although only the expansion of the components of the tip of the screw is discussed above, it should be understood that by attaching the outer surface of the expanded portion of the mandrill (24) to the inner surface of the hollow portion (26) of the screw the components (28) of the threaded portion (14) of the screw may be contracted by pushing urging the mandrill in a direction distal to the screw head (12).

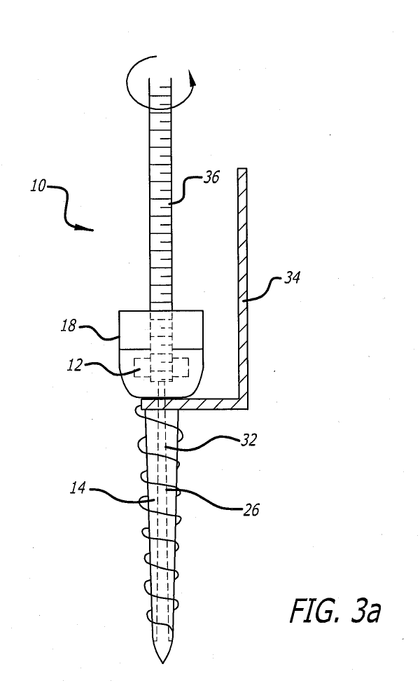

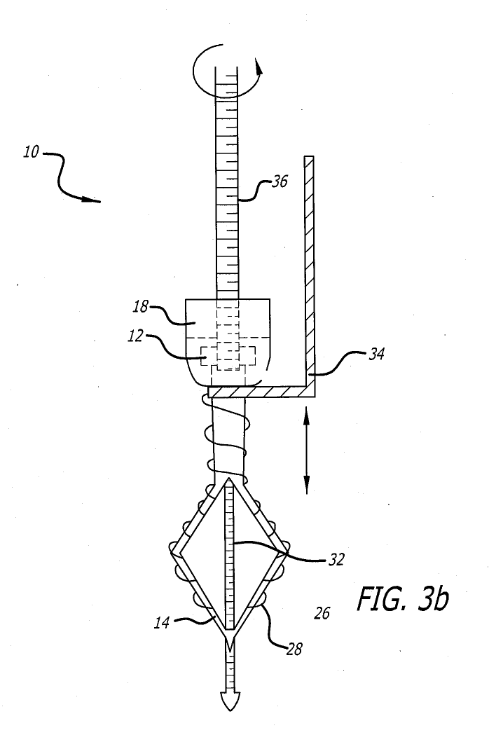

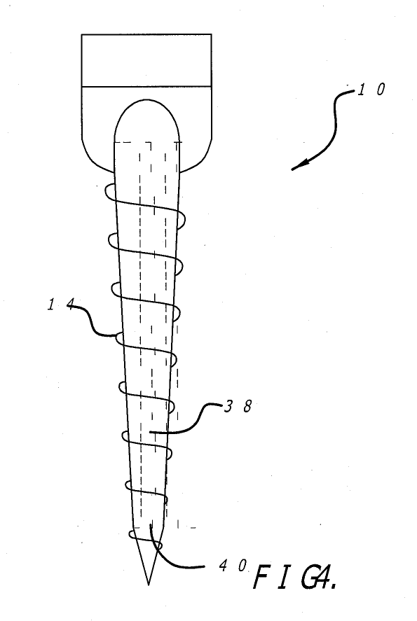

In a second exemplary embodiment, as shown in FIGS. 3a and 3b, the expansion device of the current invention employs a “molly bolt” design. As shown in such an embodiment, a rotatory braking mechanism (32) is positioned just underneath the rod seating portion (18) that allows the main thread portion to be immobilized against further rotation by an external holding mechanism (34), such as a wrench or a pin, but that permits the head of the screw (12) to rotate an internal threaded shaft (35) located within the hollow portion (26) of the screw such that the divided components (28) of the external threaded portion (14) of the screw can be expanded outward or contracted inward depending on the direction of rotation applied to the internal threaded shaft. Although a collar (32) is shown in FIGS. 3a and 3b as the rotatory braking mechanism, it should be understood that any mechanism capable of selectively immobilizing the external threaded shaft from rotating as the head of the screw is turned while allowing the internal shaft of the screw to turn may be used in the current invention, such as, for example, a clutch, ratchet, collar, etc. In operation, after the screw is inserted into the bone during which both the external and internal threaded shafts are rotated simultaneously in a first direction (with clockwise rotation in this case), a holding mechanism, such as, for example a wrench (34) is positioned against the rotatory braking mechanism (32) to hold the shank or threaded portion of the screw against further rotation, as shown in FIG. 3a. The screwdriver (36) is then engaged to rotate the head (12) of the screw (in this case in a counter-clockwise direction as shown in FIG. 3b) driving the components (28) of the threaded portion (14) of the screw upward relative to an internally threaded shaft (35) forcing the divided components of the threaded portion of the screw outward against the internal walls of the vertebral body. Although in the embodiment shown in FIGS. 3a and 3b the internal shaft is rotated in a direction opposite to the initial driving direction to expand the components of the tip of screw, it should be understood that other rotatory braking mechanisms might be employed that would allow the engagement of the internal threaded shaft in the same direction as that used to drive the external threaded shaft into the bone.

Although the above discussion has focused only on the expansion of the threaded portion of the screw, as will be understood, to reverse the expansion a surgeon would simply need to reverse the rotation of the internal threaded portion, which would in turn contract the divided components of the threaded portion of the screw. Such reversibility is essential since in osteoporotic patients it is often necessary to perform revision surgeries if a patient's condition deteriorates. In addition, such reversibility allows for removal of the device should complications arise during the procedure.

Although the above discussion has focused on the mechanical components of the screw, it should be understood that the current invention also contemplates the incorporation of osseous integration materials to assist in the fixation of the screw into the vertebral body. Such fixation material can take two basic forms. First, as previously discussed the screw (10) of the current invention may be made hollow and/or porous (38), as shown in FIG. 4, to allow for the pressure injection of materials into the spaces around the screw to facilitate the osseous integration of the device. Such materials may include any suitable fixative, such as cement, or osseous integration materials, such as, for example, calcium hydroxyapatite or calcium phosphate. Alternatively, the entire or a portion of the screw can be coated with an osseous integration material, such as, for example, calcium hydroxyapatite and calcium phosphate.

All of the above components, including can be made of any suitable surgical material, such as, for example, stainless steel or titanium.

Finally, the inclusion of a hollow cannula (38) in the center of the screw allows for the use of the screw with percutaneous minimally invasive surgical methodologies. It should be understood that the current invention is also directed to the use of the expandable fixation screw of the current invention in such a minimally invasive surgical technique. In such an embodiment, a small incision is made and an anchor guidewire is attached to a point along the vertebral body where insertion of the screw is desired. The anchor guidewire may be positioned by any conventional means, such as, for example, by a suitable catheter. In such an embodiment, the hollow cannula is provided with an outlet (40) in the tip of the threaded shaft (14). The guidewire can be threaded through the hollow cannula (38) of the screw thereby allowing for the screw to be directed down the guidewire through the opening in the patient to the insertion point without the surgeon needing to confirm the placement of the screw visually. Once in position the screw can then be operated as set forth above.

Although specific embodiments and exemplary embodiments are disclosed herein, it is expected that persons skilled in the art can and will design alternative reversible expandable fixation devices and methods that are within the scope of the following claims either literally or under the Doctrine of Equivalents.

What is claimed is:

1. An expandable internal fixation device comprising:
a screw comprised of an elongated external threaded shaft having proximal and distal ends and defining a screw axis;
a head having a driver engaging recess disposed therein arranged at a proximal end of the shaft and a tapered tip arranged at the distal end of the external threaded shaft;
a rod coupling element through which said screw may be inserted; and
wherein at least the tip of the external threaded shaft is divided into at least two components such that the components of the external threaded shaft are radially reversibly expandable.

2. The expandable internal fixation device of claim 1, wherein the external threaded shaft has a hollow central passage, and wherein an expansion device is inserted through the central passage from the head to the tapered tip of the elongated threaded shaft to reversibly expand the components of the external threaded shaft.

3. The expandable internal fixation device of claim 2, wherein the expansion device comprises a mandrill slidably mounted within said central passage, such that the components of the external threaded shaft are expanded radially outward when the mandrill is moved proximally within said passage.

4. The expandable internal fixation device of claim 3, wherein the mandrill is cooperatively threaded with the internal wall of the central passage.

5. The expandable internal fixation device of claim 3, wherein at least a portion of the mandrill is attached to the inner surface of the external threaded shaft such that the components of the external threaded shaft are expanded radially outward when the mandrill is moved proximally within said passage and contracted radially inward when the mandrill is moved distally within said passage.

6. The expandable internal fixation device of claim 2, wherein the mandrill is deformable such that any portion of the mandrill that extends above the head of the screw may be deformed to conform with the boundaries of said head.

7. The expandable internal fixation device of claim 2, wherein the screw further comprises a second internal threaded shaft disposed within the central passage and cooperatively engaged with the tip of said external threaded shaft;
a rotatory braking mechanism disposed on the external threaded shaft distally adjacent to the head of the screw such that fixing the rotatory braking mechanism in place prevents rotation of the external threaded shaft but allows the rotation of the head and internal threaded shaft; and
wherein the components of the external threaded shaft are expanded or contracted radially outward when the internal threaded shaft is rotated in isolation of the external threaded shaft.

8. The expandable internal fixation device of claim 7, wherein the internal shaft is threaded in a direction opposite the threading of the external threaded shaft.

9. The expandable internal fixation device of claim 7, wherein the rotatory braking mechanism is a collar having a wrench engaging surface.

10. The expandable internal fixation device of claim 2, wherein the hollow central passage has at least one opening in the external surface of the external threaded shaft.

11. The expandable internal fixation device of claim 10, wherein at least one of the at least one openings in the external surface of the external threaded shaft is disposed at the distal tip of the screw.

12. The expandable internal fixation device of claim 1, wherein at least a portion of the outer surface of the external threaded shaft is coated with an osseous integration material.

13. The expandable internal fixation device of claim 12, wherein the osseous integration material is selected from the group consisting of calcium hydroxyapatite and calcium phosphate

14. The expandable internal fixation device of claim 1, wherein the head is formed in a T-shape.

15. The expandable internal fixation device of claim 1, wherein the threaded shaft is divided into at least three components.

16. An expandable internal fixation device comprising:
a screw comprised of an elongated external threaded shaft having proximal and distal ends and defining a screw axis;
a head having a driver engaging recess disposed therein arranged at a proximal end of the shaft and a tapered tip arranged at the distal end of the external threaded shaft;
a rod coupling element through which said screw may be inserted;
wherein at least the tip of the external threaded shaft is divided into at least two components such that the components of the external threaded shaft are radially reversibly expandable;
wherein the external threaded shaft has a hollow central passage, and wherein a mandrill is slidably mounted within said central passage, and wherein at least a portion of the mandrill is attached to the inner surface of the external threaded shaft such that the components of the external threaded shaft are expanded radially outward when the mandrill is moved proximally within said passage and contracted radially inward when the mandrill is moved distally within said passage.

17. An expandable internal fixation device comprising:
a screw comprised of an elongated external threaded shaft having proximal and distal ends and defining a screw axis;
a head having a driver engaging recess disposed therein arranged at a proximal end of the shaft and a tapered tip arranged at the distal end of the external threaded shaft;
a rod coupling element through which said screw may be inserted;
wherein at least the tip of the external threaded shaft is divided into at least two components such that the components of the external threaded shaft are radially reversibly expandable; and
wherein the external threaded shaft further includes:
a hollow central passage and a second internal threaded shaft disposed within the central passage and cooperatively engaged with the tip of said external threaded shaft,
a rotatory braking mechanism disposed on the external threaded shaft distally adjacent to the head of the screw such that fixing the rotatory braking mechanism in place prevents rotation of the external threaded shaft but allows the rotation of the head and internal threaded shaft, and
wherein the components of the external threaded shaft are expanded or contracted radially outward when the internal threaded shaft is rotated in isolation of the external threaded shaft.

18. A method for internally fixing the cervical, thoracic or lumbar regions of the body comprising:
providing an expandable internal fixation device comprising:
a screw comprised of an elongated external threaded shaft having proximal and distal ends and defining a screw axis,
a head having a driver engaging recess disposed therein arranged at a proximal end of the shaft and a tapered tip arranged at the distal end of the external threaded shaft,
a rod coupling element through which said screw may be inserted, and
wherein at least the tip of the external threaded shaft is divided into at least two components such that the components of the external threaded shaft are radially reversibly expandable;
inserting the screw into a vertebral; and
expanding the tip of the external threaded shaft to engage the surrounding vertebral material.

19. The method of claim 18, wherein the screw is inserted into the vertebral body such that the tip of the screw is distal to the distal end of the pedicle distal such that the expanded tip engages the distal end of the pedicle.

20. The method of claim 18, wherein the external threaded shaft has a hollow central passage, and wherein the hollow central passage has at least one opening in the external surface of the external threaded shaft; and
where the method further comprises injecting an osseous integration material into the vertebral body through the hollow central passage.

21. The method of claim 20, wherein the osseous integration material is selected from the group consisting of calcium hydroxyapatite and calcium phosphate

22. The method of claim 18, wherein the external threaded shaft has a hollow central passage, and wherein a mandrill is slidably mounted within said central passage, such that the components of the external threaded shaft are expanded radially outward when the mandrill is moved proximally within said passage; and
where the method further comprises moving the mandrill proximally within the hollow passage.

23. The method of claim 18, wherein the external threaded shaft further includes:
a hollow central passage and a second internal threaded shaft disposed within the central passage and cooperatively engaged with the tip of said external threaded shaft, and
a rotatory braking mechanism disposed on the external threaded shaft distally adjacent to the head of the screw such that fixing the rotatory braking mechanism in place prevents rotation of the external threaded shaft but allows the rotation of the head and internal threaded shaft;
wherein the method further comprises fixing the external threaded shaft against rotation; and
engaging the head of the screw to rotate the internal threaded shaft in isolation of the external threaded shaft to expand the components of the tip radially outward.

24. The method of claim 18, wherein the external threaded shaft has a hollow central passage, and wherein the hollow central passage has an opening in the external surface of the tip of the external threaded shaft; and
wherein prior to inserting the device into vertebral body the method further comprises:
anchoring a guidewire onto the vertebral body at the point of insertion,
threading the guidewire through the hollow central passage, and
guiding the device to the insertion point along said guidewire.

25. A method for internally fixing the cervical, thoracic or lumbar regions of the body comprising:
providing an expandable internal fixation device comprising:
a screw comprised of an elongated external threaded shaft having proximal and distal ends and defining a screw axis, wherein the external threaded shaft has a hollow central passage, and wherein the hollow central passage has an opening in the external surface of the tip of the external threaded shaft,
a head having a driver engaging recess disposed therein arranged at a proximal end of the shaft and a tapered tip arranged at the distal end of the external threaded shaft,
a rod coupling element through which said screw may be inserted, and
wherein at least the tip of the external threaded shaft is divided into at least two components such that the components of the external threaded shaft are radially reversibly expandable;
anchoring a guidewire onto the vertebral body at the point of insertion;
threading the guidewire through the hollow central passage;
guiding the device to the insertion point along said guidewire inserting the screw into a vertebral; and
expanding the tip of the external threaded shaft to engage the surrounding vertebral material.
